(19) United States
(12) Patent Application Publication
(10) Pub. No.: US 2026/0146441 A1
(43) Pub. Date: May 28, 2026

(54) MOBILE STRUCTURE HAVING MEDICAL SCANNING DEVICE AND METHOD OF ASSEMBLING SAME

(71) Applicant: CGS Premier, Inc., Muskego, WI (US)

(72) Inventors: Benjamin Robert Schumann, Racine, WI (US); Bradley Raymond Thomas, Wind Lake, WI (US); Gregory Richard Peterson, Oconomowoc, WI (US)

(21) Appl. No.: 19/402,025

(22) Filed: Nov. 26, 2025

Related U.S. Application Data
(60) Provisional application No. 63/725,937, filed on Nov. 27, 2024.

(51) Int. Cl. E04B 1/343

(52) U.S. Cl. CPC E04B 1/34347; E04B 1/34352; E04B 1/34384

(57) ABSTRACT

A method of assembling a mobile structure having a medical scanning device includes locating a first portion of the mobile structure on a ground surface, the first portion of the mobile structure having the medical scanning device mounted therein. The method includes securing the first portion to the ground surface. The method includes positioning a second portion of the mobile structure relative to the first portion such that an open end of the first portion is positioned adjacent to an open end of the second portion. The method includes operating a winch to draw the second portion into abutment with the first portion such that the first portion and the second portion form an enclosed space defined by the first portion and the second portion. The method includes securing the second portion to the ground surface.

### CROSS-REFERENCE TO RELATED APPLICATIONS

This application claims priority to U.S. Provisional Application No. 63/725,937, filed Nov. 27, 2024, which is hereby incorporated by reference in its entirety.

### FIELD OF INVENTION

The present disclosure relates to mobile medical facilities, and more particularly to a mobile radiation oncology system comprising two separate transportable sections that are assembled onsite.

### BACKGROUND

Mobile medical facilities have become increasingly valuable for providing specialized healthcare services to underserved areas or as supplemental capacity for existing medical institutions. These facilities enable healthcare providers to deliver advanced medical treatments and diagnostic services directly to communities that may lack permanent infrastructure or face geographic barriers to accessing care.

Radiation oncology represents one area where mobile medical facilities have shown particular promise. Radiation therapy equipment, such as linear particle accelerators (LINACs), can provide life-saving cancer treatments but requires substantial infrastructure investment that may not be feasible for all healthcare facilities. Mobile radiation oncology systems allow healthcare providers to extend these services to multiple locations using a single equipment investment.

Traditional mobile medical facilities are typically constructed as single-unit trailer systems that are transported via truck and positioned at treatment sites. These conventional designs often present challenges related to patient accessibility, as the elevated trailer configuration requires patients to navigate steep ramps or stairs to enter and exit the facility. Such access requirements can pose difficulties for elderly patients, individuals with mobility limitations, or patients requiring wheelchair or stretcher transport.

The transportation of large medical equipment also presents logistical challenges due to Department of Transportation regulations governing vehicle dimensions, weight limits, and height restrictions. These regulatory constraints can limit the size and configuration of mobile medical facilities, potentially affecting the interior space available for equipment placement and patient care activities.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a method of assembling a mobile structure having a medical scanning device is provided. The method comprises locating a first portion of the mobile structure on a ground surface, where the first portion of the mobile structure has the medical scanning device mounted therein. The method further comprises securing the first portion to the ground surface. The method also comprises positioning a second portion of the mobile structure relative to the first portion such that an open end of the first portion is positioned adjacent to an open end of the second portion. The method additionally comprises operating a winch to draw the second portion into abutment with the first portion such that the first portion and the second portion form an enclosed space defined by the first portion and the second portion. The method further comprises securing the second portion to the ground surface.

According to other aspects of the present disclosure, the method may include one or more of the following features. The ground surface may be a poured slab, and securing the first portion to the ground surface may include providing a fastener through a mounting anchor coupled to the first portion and into the poured slab. Securing the second portion to the ground surface may include providing a fastener through a mounting anchor coupled to the second portion and into the poured slab. The mobile structure may include a floor surface upon which a user is able to walk within the enclosed space, and the winch may be located below the floor surface. The winch may be coupled to one of the first portion or the second portion and an attachment point may be coupled to the other of the first portion or the second portion, and operating the winch may include coupling a cable of the winch to the attachment point. The attachment point may be located below the floor surface. The method may further comprise positioning a first guide plate on a first wall of the first portion and a second guide plate on a second wall of the first portion, and the first and second guide plates may guide a first wall and a second wall of the second portion towards the first wall and the second wall of the first portion during operation of the winch. The first guide plate may extend outward from an end of the first wall of the first portion at an angle of 15-45 degrees and a distance of 4-12 inches. The method may further comprise aligning the first portion and the second portion with a chain binder. The method may further comprise coupling casters to the second portion and moving the second portion towards the first portion on the casters prior to operating the winch. The method may further comprise removing the casters prior to operating the winch. The method may further comprise removing the first portion from a wheeled trailer prior to locating the first portion of the mobile structure on the ground surface and removing the second portion from the wheeled trailer prior to positioning the second portion of the mobile structure relative to the first portion. Locating the first portion of the mobile structure on the ground surface may include locating the first portion within a bunker having sidewalls that surround at least three sides of the first portion. The bunker may have sidewalls having a thickness of at least two feet and a height of at least twelve feet. Locating the first portion of the mobile structure on the ground surface may include locating the first portion directly on the ground surface with no intervening structure.

According to another aspect of the present disclosure, a mobile structure having a medical scanning device is provided. The mobile structure comprises a first portion having a floor surface and a plurality of walls, where the medical scanning device is mounted to the floor surface of the first portion, and the first portion is configured to be transported on a trailer. The mobile structure further comprises a second portion having a floor surface and a plurality of walls, where the second portion is formed separate from the first portion and is configured to be transported on a trailer separate from the first portion. The mobile structure additionally comprises a winch located below the floor surface of the first portion, where the winch has a cable configured to couple to an attachment point of the second portion, and the winch is configured to draw the second portion into abutment with the first portion.

According to other aspects of the present disclosure, the mobile structure may include one or more of the following features. The mobile structure may further comprise a first mounting anchor coupled to the first portion and a first fastener configured to extend through the first mounting anchor to couple the first portion to a poured slab, and a second mounting anchor coupled to the second portion and a second fastener configured to extend through the second mounting anchor to couple the second portion to the poured slab. The mobile structure may further comprise a first guide plate coupled to a first wall of the plurality of walls of the first portion and a second guide plate coupled to a second wall of the plurality of walls of the first portion, and the first guide plate and the second guide plate may be configured to align the plurality of walls of the first portion with the plurality of walls of the second portion. The mobile structure may further comprise caster wheels coupled to the second portion such that the second portion is movable relative to the first portion.

According to a further aspect of the present disclosure, a mobile structure having a medical scanning device is provided. The mobile structure comprises a first portion and a second portion collectively forming an enclosed space configured to support the medical scanning device, a patient, and an operator of the medical scanning device.

Any of the elements described above may be used in combination with one another to provide various configurations of the mobile structure and assembly methods. The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1A illustrates an isometric view of a mobile structure with a first module and a second module in an assembled configuration.

FIG. 1B illustrates a detailed view of a sidewall with an attachment point.

FIG. 2 illustrates a top sectional view of the mobile structure showing a winch system for drawing the second module toward the first module.

FIG. 3 illustrates a top view of the mobile structure of FIG. 2 with the modules in abutment.

FIG. 4A illustrates an isometric view of the mobile structure of FIG. 1A with the modules in a separated configuration.

FIG. 4B depicts a detailed view of an attachment point and an opening configured to receive the attachment point.

FIG. 4C illustrates a detail view of a portion of the first module showing one of the D-rings for chain binder attachment.

FIG. 4D illustrates a chain binder used in the alignment process of the mobile structure.

FIG. 4E illustrates a detailed view of a guide plate.

FIG. 5A illustrates a side view of the mobile structure of FIG. 4A showing the modules in a separated configuration.

FIG. 5B illustrates a section view of the first module taken along section line D-D.

FIG. 5C illustrates a section view of the second module taken along section line E-E.

FIG. 5D illustrates a detailed side view of a base of the first module showing an opening for a cable of the winch.

FIG. 5E illustrates a detailed side view of the second module showing a base with an opening for a D-ring.

FIG. 6A illustrates a perspective view of the mobile structure showing a connection interface between the first module and the second module.

FIG. 6B depicts a detailed view of a corner sealing plate.

FIG. 6C illustrates a detailed view of a portion of a side sealing plate at the interface between the modules.

FIG. 7A illustrates an end view of the mobile structure of FIG. 6A.

FIG. 7B illustrates a section view of the mobile structure of FIG. 7A taken along section line K-K.

FIG. 7C illustrates a detailed sectional view of the interface between the first module and the second module.

FIG. 8 illustrates a perspective view of a ground installation site prepared for receiving the mobile structure.

FIG. 9 illustrates a perspective view of the first module positioned on a removable gooseneck trailer backed into a bunker.

FIG. 10 illustrates an isometric view of the first module being lifted vertically off the trailer.

FIG. 11 illustrates the first module of FIG. 10 in its final position on a ground surface within the bunker.

FIG. 12 illustrates a perspective view of the mobile structure during an intermediate stage of assembly with the second module being transported on a trailer.

FIG. 13 illustrates the mobile structure of FIG. 12 with the second module lifted off the trailer.

FIG. 14 illustrates the mobile structure of FIG. 13 with caster wheels installed on the second module.

FIG. 15 illustrates an isometric view of the mobile structure of FIG. 14 with the second module lifted to remove the caster wheels.

FIG. 16 illustrates an isometric view of the mobile structure in its assembled configuration positioned on the ground surface.

FIG. 17 illustrates the mobile structure of FIG. 16 with additional exterior components including a ramp, awning, and sheathing.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The design of the mobile structure 200 addresses specific constraints imposed by Department of Transportation (DOT) regulations that govern the transport of oversized loads on public roadways. The regulatory requirements lead the design of the mobile structure to a two-module configuration and influence the selection of transport equipment and dimensional specifications for the mobile radiation oncology system.

Weight limitations under DOT regulations establish maximum allowable loads for commercial transport vehicles. The first module 204 weighs approximately 50,000 pounds when fully equipped with a medical scanner 300 (i.e., medical scanning device) and associated shielding materials. The second module 232 weighs approximately 30,000 pounds, including the control equipment, patient preparation areas, and structural components. The separation of the mobile structure 200 into two distinct modules allows each module to remain within acceptable weight limits for highway transport while distributing the total system weight across separate transport operations.

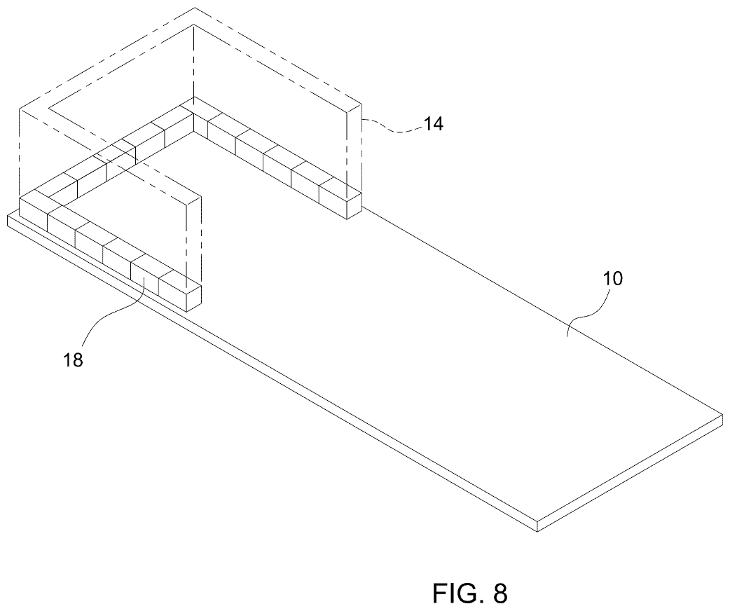

Dimensional restrictions under DOT regulations impose length limitations on trailer configurations. A removable gooseneck trailer 112 (FIG. 8) provides the transport platform for each module, with the removable gooseneck trailer 112 having a maximum length of 29.5 feet. The first module 204 has a length of 29 feet to comply with this transport limitation, while the second module 232 has a length of 26 feet. The removable gooseneck trailer 112 includes a gooseneck 116 that connects to the tractor unit, a main deck 120 that supports the transported module, and a rear deck 124 that provides additional structural support. The trailer assembly rides on wheels 128 that distribute the load during transport operations.

Height requirements under DOT regulations establish maximum vertical clearances for transport vehicles traveling on public roadways. The removable gooseneck trailer 112 configuration maintains the overall transport height below regulatory thresholds by positioning the transported modules closer to the roadway surface compared to conventional trailer designs. This low-profile transport approach accommodates the substantial height of each module while remaining within DOT compliance parameters.

The assembled mobile structure 200 achieves an overall length of approximately 58 feet when the first module 204 and second module 232 are joined together at the installation site. The mobile structure 200 has an overall width of approximately 12 feet in the standard configuration. When an extension portion is deployed, the overall width expands to approximately 16 feet, providing additional interior space for patient movement and equipment operation. These final dimensions exceed what would be permissible for highway transport as a single unit, confirming the necessity of the two-module approach for regulatory compliance.

The modules are transported on dedicated low boy semi-trailers that provide the structural platform and mobility for highway transport. The low boy configuration positions the transported modules at a reduced height above the roadway, contributing to overall height compliance while accommodating the substantial vertical dimensions of the radiation shielding and medical equipment housed within each module.

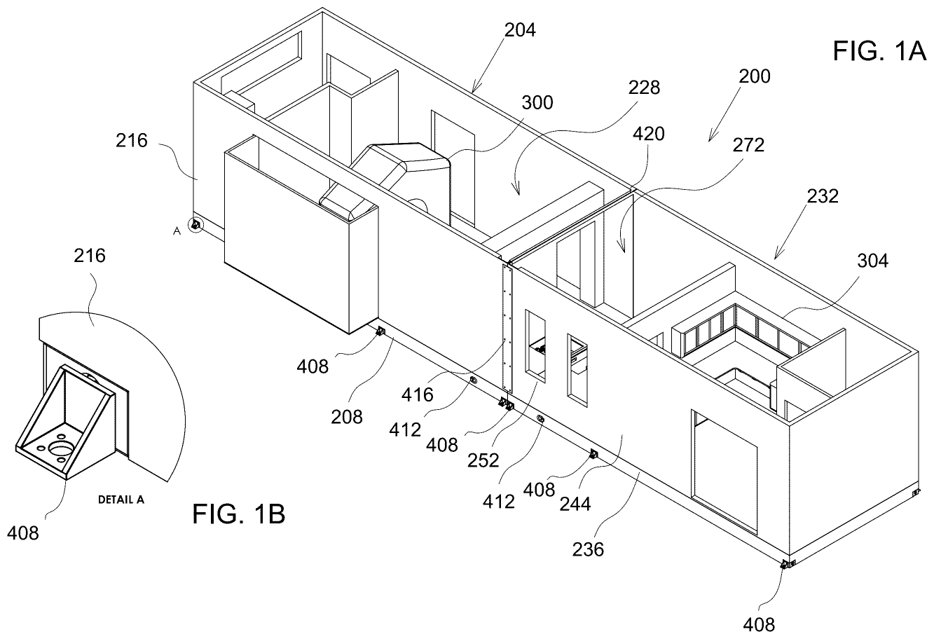

Referring to FIG. 1A, the mobile structure 200 includes a first module 204 and a second module 232 configured to connect together to form a complete radiation oncology facility. The first module 204 includes a base 208 that provides structural support and houses the medical scanner 300. Sidewalls 216 extend upward from the base 208 to define the perimeter of the first module 204. An interior 228 within the first module 204 accommodates the medical scanner 300 and provides space for patient positioning and treatment procedures.

The second module 232 includes a base 236 that forms the structural foundation of the module. Sidewalls 244 extend upward from the base 236 to define the exterior boundaries of the second module 232. A window 252 is positioned within one of the sidewalls 244 to provide natural lighting to the interior spaces. An interior 272 within the second module 232 houses other office equipment and medical device control devices 304 that enable operators to control the medical scanner 300 and manage patient treatment protocols.

The mobile structure 200 includes multiple interior spaces within the second module 232 to support comprehensive patient care and operational functions. An electrical room houses power distribution systems and electrical infrastructure for the medical scanner 300 and associated equipment. A control room contains the other office equipment and medical device control devices 304 that allow operators to monitor and control radiation treatment procedures. In some embodiments, a changing room provides privacy for patients to change into appropriate garments for medical procedures and/or a restroom offers patient convenience and accessibility during treatment visits.

The interior 228 of the first module 204 and the interior 272 of the second module 232 collectively provide ample space to maneuver patients in wheelchairs or on stretchers throughout the mobile structure 200. The floor surfaces, sidewalls 216, 244, roof structures, and doors of both modules are lined with lead shielding to provide radiation protection for patients, operators, and surrounding areas during operation of the medical scanner 300.

As described in greater detail below, an interface 420 defines the connection zone where the first module 204 and second module 232 join together during assembly, multiple attachment points 408 are positioned along the lengths of both modules to facilitate the connection process, and a coupling panel 416 provides one element used to secure the first module 204 to the second module 232 at the interface 420.

The mobile structure 200 forms an enclosed space when the first module 204 and second module 232 are assembled together. The enclosed space supports the medical scanner 300, accommodates patients during treatment procedures, and provides workspace for operators of the medical scanner 300. The first module 204 and second module 232 each have a floor surface and a plurality of walls that define their respective interior spaces. The medical scanner 300 mounts to the floor surface of the first module 204 within the interior 228. Each module is configured to be transported on a trailer separate from the other module, with the first module 204 transported on one removable gooseneck trailer 112 and the second module 232 transported on a separate removable gooseneck trailer 112.

Referring to FIG. 1B, the attachment point 408 couples to a sidewall 216 of the mobile structure 200, or more specifically along a side surface of the base 208 that is positioned below and aligned with the sidewall 216. While the attachment point identified in FIG. 1B corresponds to the first module 204, each of the first and second modules 204, 232 include multiple attachment points 408 about the perimeter of the structure. The attachment point 408 is positioned on the exterior surface of the mobile structure 200 and extends outward from the sidewall 216 to provide a mounting interface for connecting components during the assembly process. The structural configuration of the attachment point 408 includes a mounting portion that secures to the base 208 and sidewall 216 and a projecting portion that extends away from the exterior surface to create a coupling location.

The sidewall 216 and base 208 form part of the structural framework of the mobile structure 200 and one or both provide support for the attachment point 408. The attachment point 408 mounts directly to the exterior surface, creating a secure connection point that can withstand the forces applied during assembly operations. The attachment point 408 serves as a coupling location for connecting various components during the assembly process of the mobile structure 200. The projecting configuration of the attachment point 408 provides clearance from the sidewall 216 surface, allowing assembly equipment to engage with the attachment point 408 without interference from the sidewall 216 structure. In some embodiments, the attachment point 408 accommodates the connection of lifting equipment, alignment devices, and other assembly tools that facilitate the positioning and joining of the first module 204 and second module 232. Caster wheels 404 are mountable to the attachment points 408 to permit rolling movement of the respective module 204, 232 along a ground surface 10. The structural mounting of the attachment point 408 to the sidewall 216 distributes applied loads across the sidewall 216 structure, preventing localized stress concentrations that could compromise the integrity of either the attachment point 408 or the sidewall 216.

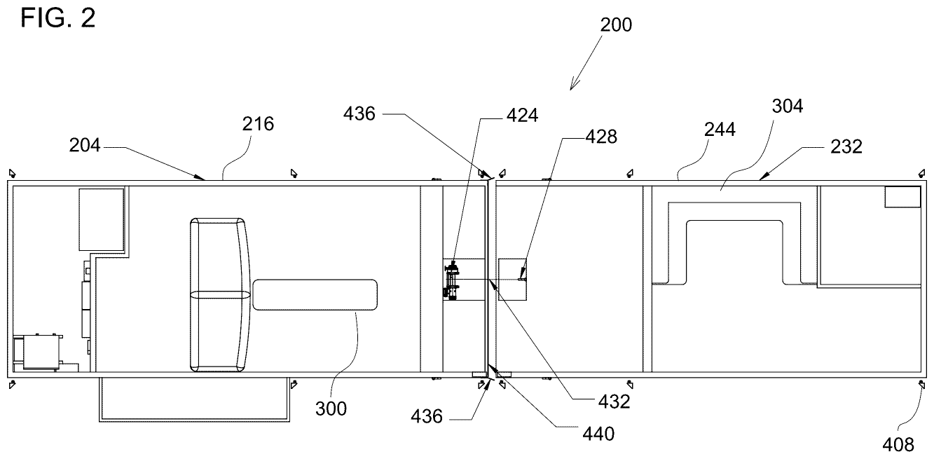

Referring to FIG. 2, the mobile structure 200 includes an internal arrangement that facilitates the assembly of the first module 204 and second module 232 through a mechanical drawing system. A winch 424 is positioned within the first module 204 and provides the motive force to draw the second module 232 toward the first module 204 during assembly operations. The winch 424 is located below a floor surface of the first module 204, positioning the winch 424 beneath the area upon which a user is able to walk within the enclosed space formed by the assembled mobile structure 200.

A cable 432 extends from the winch 424 toward the second module 232 to establish the mechanical connection between the two modules during assembly. The cable 432 is configured to couple to an attachment point 408 located in the second module 232. The attachment point 408 in the second module 232 includes a D-ring 428 that provides a coupling location for the cable 432. The D-ring 428 is positioned below the floor surface of the second module 232, beneath the operational floor level of the assembled mobile structure 200.

The winch 424 operates by retracting the cable 432, thereby drawing the second module 232 toward the first module 204 until the modules are in abutment at the interface 420. The positioning of the winch 424 below the floor surface creates a mechanical advantage by reducing drag forces that would be present if the winch 424 were located at a higher position. The low mounting position of the winch 424 also maintains the mechanical components out of the operational space within the assembled mobile structure 200.

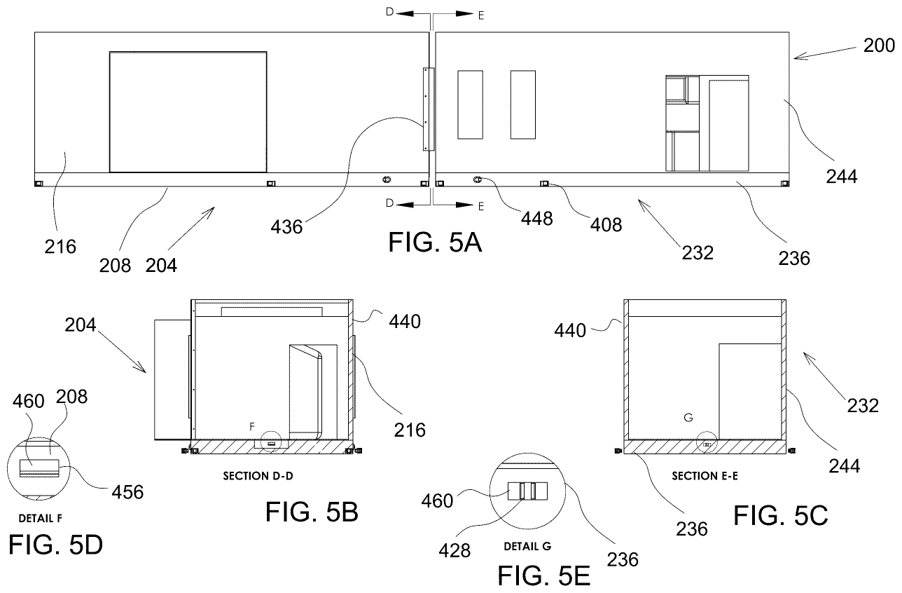

A gasket 440 is positioned at the interface 420 between the first module 204 and the second module 232. The gasket 440 provides a seal between the two modules when the winch 424 draws the modules together into abutment. The gasket 440 creates a weather tight seal at the junction between the first module 204 and second module 232, contributing to the formation of the enclosed space within the assembled mobile structure 200, and is shown in greater detail in FIGS. 5B and 5C.

Guide plates 436 are mounted on the sidewalls 216 of the first module 204 and extend outward at an angle to facilitate alignment of the second module 232 with the first module 204 during the assembly process. The guide plates 436 assist in directing the sidewalls 244 of the second module 232 into proper alignment with the sidewalls 216 of the first module 204 as the winch 424 pulls the modules together. The angled configuration of the guide plates 436 provides guiding surfaces that direct the second module 232 into the correct position relative to the first module 204, ensuring proper alignment of the interface 420 between the two modules.

The mechanical drawing system enables one step of the assembly method by providing controlled movement of the second module 232 toward the first module 204, especially when the two modules 204, 232 are in close proximity to one another. The winch 424 coupled to the first module 204 and the attachment point 408 coupled to the second module 232 establish the mechanical connection that allows the winch 424 to draw the second module 232 into abutment with the first module 204.

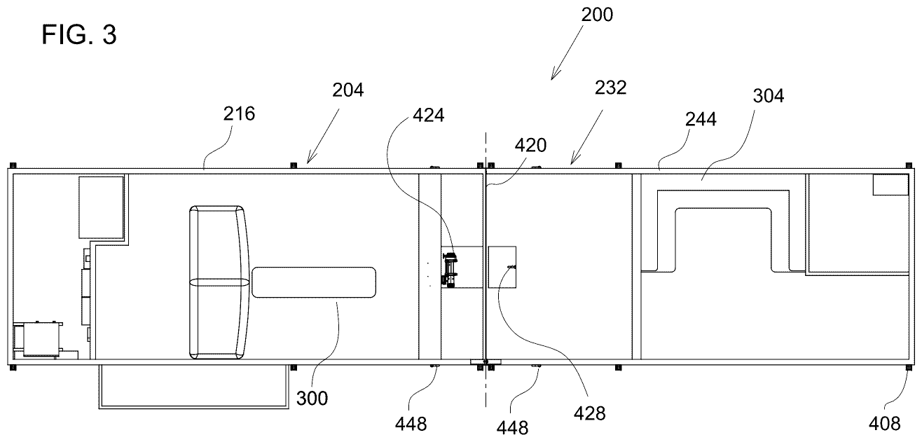

Referring to FIG. 3, the mobile structure 200 is shown in a fully assembled configuration where the first module 204 and the second module 232 have been drawn together such that the interface 420 between the two modules is sealed. The top view demonstrates the spatial arrangement of the components and the relationship between the first module 204 and the second module 232 when connected. The winch 424 has completed the drawing operation, positioning the first module 204 and second module 232 in direct abutment at the interface 420.

The assembled configuration shows the medical scanner 300 positioned within the first module 204 and the other office equipment and medical device control devices 304 housed within the second module 232. The interface 420 forms a continuous junction between the two modules, with the gasket 440 providing environmental sealing along the connection zone.

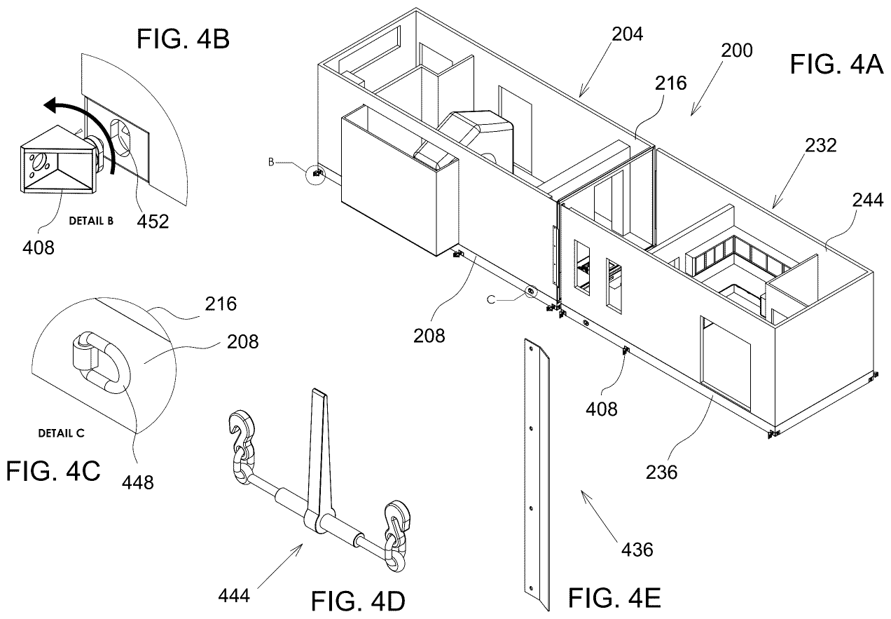

As described in greater detail with respect to FIGS. 4C and 4D, exterior D-rings 448 are positioned on the exterior surfaces of both the first module 204 and the second module 232, providing additional alignment points during the assembly process. The D-rings 448 are located on the sidewalls of each module and serve as attachment points for alignment equipment used during the joining of the two modules. The positioning of the D-rings 448 on both modules allows for the connection of chain binders 444 (FIG. 4D) or other tensioning devices that assist in drawing the modules together and maintaining proper alignment during the assembly sequence. In some embodiments, a heavy duty ½ inch thick grade 80 transport chain is used in conjunction with the D-rings 448 and the chain binder 444.

The D-rings 448 complement the operation of the winch 424 by providing supplementary alignment control during the assembly process. The D-rings 448 are accessible from the exterior of the mobile structure 200, allowing assembly personnel to attach alignment equipment without requiring access to the interior spaces of either module. The strategic placement of the D-rings 448 on both the first module 204 and second module 232 enables balanced force application during alignment operations, contributing to the precise positioning of the modules relative to one another at the interface 420.

Referring to FIG. 4A, the mobile structure 200 is shown in a separated configuration where the first module 204 and the second module 232 are positioned apart from one another by approximately 8-12 inches. The separated configuration illustrates the relationship between the modules prior to final assembly and provides access to the connection hardware that facilitates the joining process. The first module 204 includes the base 208 and sidewalls 216 that define the structural framework of the module. The second module 232 includes the base 236 and sidewalls 244 that form the exterior boundaries of the module.

Referring to FIG. 4B, the attachment point 408 includes a mounting system that enables secure installation and removal of the attachment point 408 during assembly operations. An opening 452 is configured to receive the attachment point 408 through a rotational installation process. The attachment point 408 is inserted into the opening 452 in a rotated configuration, as shown in FIG. 4B, where the attachment point 408 is oriented to pass through the opening 452. After insertion, the attachment point 408 is rotated to an installed position where the attachment point 408 is secured within the opening 452.

The opening 452 has a non-circular shape that prevents removal of the attachment point 408 without rotation out of the installed position. The non-circular configuration of the opening 452 creates a mechanical lock that maintains the attachment point 408 in the installed position during assembly operations. The shape of the opening 452 accommodates the insertion of the attachment point 408 in the rotated configuration while preventing inadvertent removal when the attachment point 408 is in the installed position. The rotational installation system provides a secure mounting method that allows for controlled installation and removal of the attachment point 408 as needed during different phases of the assembly process.

Referring to FIG. 4C, the first module 204 includes connection hardware mounted on the exterior surfaces to facilitate alignment during assembly operations. The base 208 and/or sidewalls 216 provide structural support for the connection hardware. The D-rings 448 are mounted on the exterior surfaces of the sidewalls 216 and are positioned to receive hooks of alignment equipment during the assembly process. The D-rings 448 are coupled to the base 208 and positioned below adjacent sidewalls 216, providing attachment points that are accessible from the exterior of the first module 204.

The D-rings 448 are configured to receive hooks of a chain binder 444 (FIG. 4D) for alignment purposes during assembly of the mobile structure 200. The positioning of the D-rings 448 on the exterior surfaces allows assembly personnel to attach alignment equipment without requiring access to the interior spaces of the first module 204. The D-rings 448 provide secure attachment points that can withstand the forces applied during alignment operations while maintaining their position on the exterior surfaces of the sidewalls 216. Similar D-rings 448 are positioned on the second module 232, and on the opposite side of each of the first and second modules 204, 232 to provide corresponding attachment points for alignment equipment.

Referring to FIG. 4D, a chain binder 444 provides alignment assistance during the assembly of the mobile structure 200. The chain binder 444 is a mechanical tensioning device that includes a central body with a lever mechanism and hooks at both ends. The hooks of the chain binder 444 are configured to attach to the D-rings 448 on the exterior surfaces of the first module 204 and the second module 232. The lever mechanism of the chain binder 444 operates to apply tension between the attached D-rings 448, thereby drawing the two modules together and assisting in their alignment during assembly. The chain binder 444 works in conjunction with the winch 424 to facilitate proper positioning of the first module 204 and second module 232 relative to one another. The tensioning capability of the chain binder 444 provides supplementary force application that complements the drawing action of the winch 424. The chain binder 444 enables assembly personnel to apply controlled tension between specific points on the two modules, contributing to precise alignment of the interface 420 between the first module 204 and second module 232 transverse to the drawing direction of the winch 424 as the second module 232 is drawn towards the first module 204. The mechanical advantage provided by the lever mechanism of the chain binder 444 allows for the application of substantial tensioning forces with manageable manual effort.

Referring to FIG. 4E, the guide plate 436 includes an elongated, angled configuration that facilitates alignment of the second module 232 with the first module 204 during assembly operations. The guide plate 436 has a narrow mounting end and extends outward at an angle to a wider distal end, forming a wedge-like profile that provides a guiding surface for the approaching second module 232. The guide plate 436 includes mounting holes positioned along the narrow mounting end that facilitate attachment to the sidewalls 216 of the first module 204.

The angled configuration of the guide plate 436 provides a guiding surface that directs and aligns the second module 232 during the assembly process when the two modules are drawn together by the winch 424. The guide plate 436 extends outward from an end of a first wall of the first module 204 at an angle of 15-45 degrees and a distance of 4-12 inches. In some embodiments, the guide plate 436 extends outward approximately 8 inches at an angle of approximately 30 degrees from the sidewalls 216. The tapered profile of the guide plate 436 creates a funnel-like guiding surface that captures and directs the sidewalls 244 of the second module 232 into proper alignment with the sidewalls 216 of the first module 204. The guide plate 436 is removable after assembly is completed.

A first guide plate 436 is positioned on a first wall of the first module 204 and a second guide plate 436 is positioned on a second wall of the first module 204 (opposite the first wall). The first and second guide plates 436 guide a first wall and a second wall of the second module 232 towards the first wall and the second wall of the first module 204 during operation of the winch 424. The guide plates 436 are configured to align the plurality of walls of the first module 204 with the plurality of walls of the second module 232, ensuring proper positioning of the modules at the interface 420.

Referring to FIG. 5A, the mobile structure 200 is shown in a side view with the first module 204 and the second module 232 positioned in a separated configuration. The open ends of the two modules are positioned to face one another in preparation for assembly operations.

The guide plates 436 are positioned on opposing sidewalls 216 of the first module 204, extending outward from the ends of the sidewalls 216 at an angle. The guide plates 436 are configured to guide the sidewalls 244 of the second module 232 during the assembly process when the two modules are drawn together by the winch 424, as described above. The attachment points 408 are visible along the perimeter of both modules, providing connection locations for assembly equipment and components. The D-rings 448 for chain binder attachment are positioned on the exterior surfaces of both the first module 204 and second module 232, enabling the connection of alignment equipment during assembly operations.

Referring to FIG. 5B, a cross-sectional view of the first module 204 taken along section line D-D shows the internal structure and sealing configuration of the module. The sidewalls 216 extend upward from the base to define the interior space of the first module 204. The gasket 440 is positioned along the edges of the sidewalls 216, as well as the top wall (ceiling) and the base, providing a sealing surface that interfaces with the second module 232 when the two modules are brought into abutment during assembly. In particular, the gasket 440 extends along the edges of the walls, base, and ceiling of the first module 204 along the surfaces that abut the second module 232 when assembled. The gasket 440 creates a continuous sealing surface around the perimeter of the interface 420 between the two modules. In some embodiments, the gasket 440 is a closed cell foam that is approximately ¼ inch thick, providing compressible sealing material that accommodates minor variations in the alignment of the two modules while maintaining an effective environmental seal.

The cross-sectional view also references Detail F, shown in FIG. 5D, which shows an opening 456 within the base 208 through which the cable 432 from the winch 424 extends toward the second module 232. The opening 456 for the winch provides access for the cable 432 to pass through the base 208 of the first module 204, enabling the mechanical connection between the winch 424 and the attachment point 408 in the second module 232. As the base 208 includes the opening 456 through which the cable 432 of the winch 424 extends toward the second module 232, the opening 456 for the winch is positioned to provide access to the winch mechanism from below the floor surface of the first module 204, allowing the cable 432 to pass through the base 208 structure.

Referring to FIG. 5C, a cross-sectional side view of the second module 232 shows the internal structure and sealing configuration of the module. The gasket 440, similar to the one on the first module 204, is positioned along the edges of the sidewalls 244, base 236, and ceiling, providing sealing surfaces that interface with the first module 204 when the modules are assembled together.

A bottom surface 410 of the second module 232 is visible in the cross-sectional view and shown in greater detail in FIG. 5E. An opening is formed in the base 236 of the second module 232 through which the cable 432 from the winch 424 extends to reach the D-ring 428 located within the base 236. The opening provides access for the mechanical connection between the winch 424 in the first module 204 and the D-ring 428 positioned below the floor surface of the second module 232.

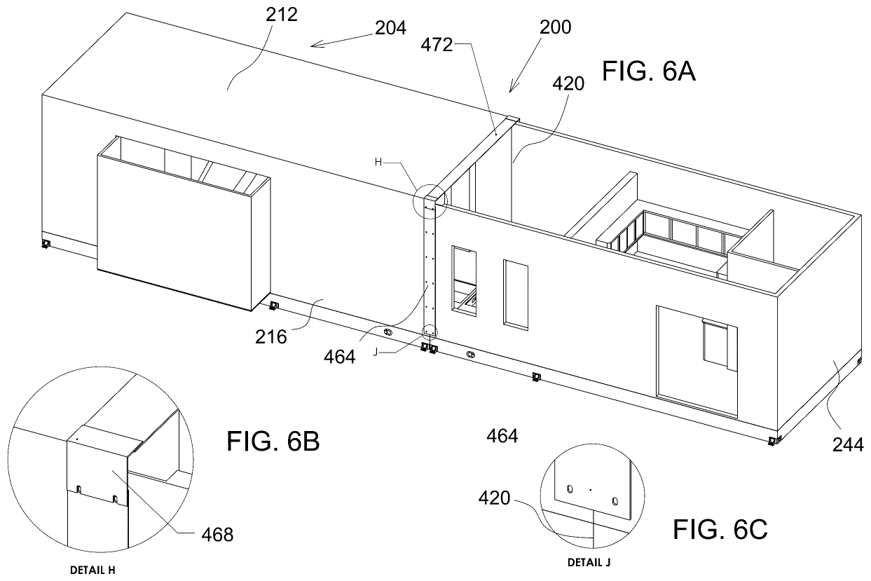

Referring to FIG. 6A, the mobile structure 200 is shown in an assembled configuration where the first module 204 and the second module 232 are joined together at the interface 420. The sidewalls 216 of the first module 204 and the sidewalls 244 of the second module 232 are aligned with one another, creating a continuous exterior surface across both modules. A roof 212 of the first module extends over the first module 204, providing structural coverage and environmental protection for the interior space. A roof of the second module 232 is omitted from this view to illustrate the interface 420.

Hardware components and a perimeter sealing system are installed to seal the two modules together after the modules have been positioned and drawn into abutment. A side sealing plate 464 is positioned along the lateral edge of the interface 420, covering the joint between the sidewalls 216 of the first module 204 and the sidewalls 244 of the second module 232. The side sealing plate 464 extends along the vertical junction between the two modules, providing structural continuity across the connection zone and environmental sealing that prevents air and moisture infiltration through the interface 420.

A top sealing plate 472 is positioned along the upper edge of the interface 420, covering the joint between the roof sections of the two modules. The top sealing plate 472 spans across the horizontal junction at the roof level, creating a continuous exterior surface that bridges the connection between the first module 204 and the second module 232. The top sealing plate 472 provides environmental sealing along the upper portion of the interface 420, preventing water infiltration and maintaining the structural integrity of the roof assembly across both modules.

The side sealing plate 464 and the top sealing plate 472 work together to create a comprehensive sealing system around the perimeter of the interface 420, along with a corner sealing plate 468 (FIG. 6B). The sealing plates provide structural continuity by bridging the gaps between the separate modules and creating a unified exterior envelope for the assembled mobile structure 200. The environmental sealing function of the sealing plates prevents the infiltration of air, moisture, and other environmental elements through the connection zone, maintaining the controlled interior environment within the assembled mobile structure 200.

Referring to FIG. 6B, a corner sealing plate 468 provides sealing coverage at the junction between the side sealing plate 464 and the top sealing plate 472. The corner sealing plate 468 has an angled configuration designed to cover the corner joint where the top sealing plate 472 and the side sealing plate 464 meet. Mounting holes are positioned along the edges of the corner sealing plate 468 for attachment to the side sealing plate 464. The corner sealing plate 468 includes openings for attaching to the top sealing plate 472 or attaches to the top sealing plate 472 through alternative mounting methods. The angled configuration of the corner sealing plate 468 accommodates the geometric transition between the vertical orientation of the side sealing plate 464 and the horizontal orientation of the top sealing plate 472. The corner sealing plate 468 eliminates gaps that would otherwise exist at the corner junction, providing complete environmental sealing around the entire perimeter of the interface 420.

Referring to FIG. 6C, the side sealing plate 464 includes mounting holes that allow the side sealing plate 464 to be coupled to the adjacent sidewalls 216, 244 of the two modules. The mounting holes within the side sealing plate 464 are positioned to align with corresponding attachment points on both the first module 204 and the second module 232, enabling secure fastening of the side sealing plate 464 across the interface 420.

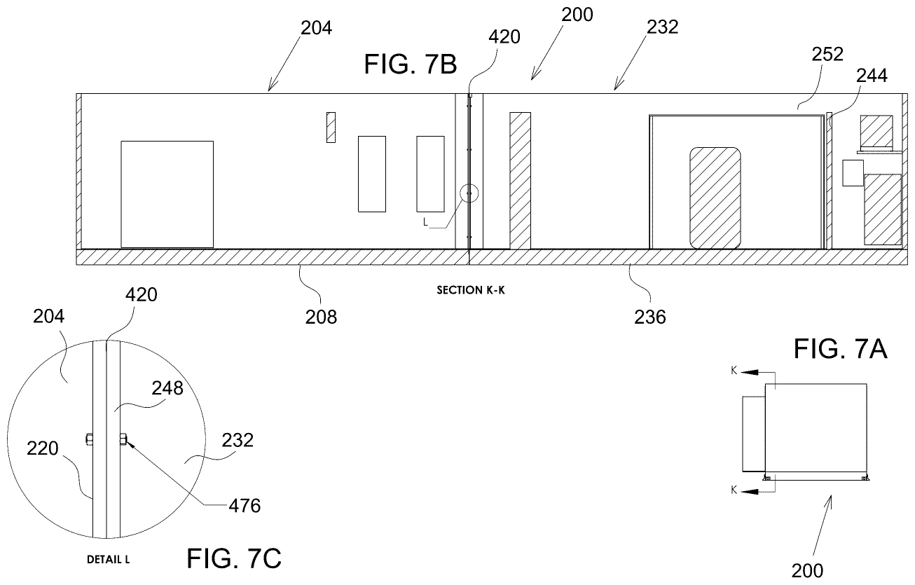

Referring to FIG. 7A, an end view of the mobile structure 200 provides the basis for understanding the sectional relationship between the first module 204 and the second module 232 at the interface 420. The end view shows the assembled mobile structure 200 from a perspective that illustrates the overall configuration and establishes the reference for the sectional view detailed in FIG. 7B.

Referring to FIG. 7B, a sectional view of the mobile structure 200 taken along section line K-K demonstrates the internal configuration at the interface 420 where the first module 204 and the second module 232 meet. The sectional view shows the structural relationship between the base 208 of the first module 204 and the base 236 of the second module 232 at their junction. The interface 420 is formed where the two mating surfaces of the modules meet, creating a continuous structural connection between the separate modules.

The sectional view illustrates how the two modules are positioned relative to one another at the interface 420, with the gasket 440 providing environmental sealing along the connection zone. The base 208 of the first module 204 aligns with the base 236 of the second module 232, creating surface continuity across the junction. The sidewalls 244 of the second module 232 align with corresponding sidewalls of the first module 204, maintaining the exterior envelope of the assembled mobile structure 200.

The sectional view demonstrates the structural integration achieved when the winch 424 draws the second module 232 into abutment with the first module 204. The interface 420 creates a continuous structural connection that unifies the two separate modules into a single functional facility. The alignment of the bases and sidewalls at the interface 420 distributes structural loads across both modules, creating a stable platform for the medical scanner 300 and other office equipment and medical device control devices 304.

Referring to FIG. 7C, a detailed sectional view of the interface 420 shows the mechanical fastening system that secures the first module 204 and the second module 232 together. A forward wall 220 of the first module 204 is positioned adjacent to a forward wall 248 of the second module 232 at the interface 420. The forward walls 220, 248 form the mating surfaces where the two modules connect, creating the structural junction between the separate modules.

A fastener 476 extends through both the forward wall 220 of the first module 204 and the forward wall 248 of the second module 232, securing the two modules together at the interface 420. The fastener 476 is a bolt that penetrates through aligned openings in both forward walls 220, 248 to mechanically join the first module 204 and the second module 232 and is secured by a nut. The fastener 476 creates a direct mechanical connection between the structural frameworks of the two modules, providing structural integrity to the assembled mobile structure 200. In some embodiments, the forward walls 248 are formed of a hollow tube construction. As such, in some embodiments, the hardware 476 is recessed within the forward wall 248 to prevent crushing the hollow steel tube structure during gasket compression.

The fastening system includes numerous aligned openings positioned throughout the forward walls 220, 248 to receive multiple fasteners 476. In some embodiments, four fasteners 476 are positioned in each wall, four fasteners 476 are positioned in the ceiling, and four fasteners 476 are positioned in the base, providing comprehensive mechanical coupling across the entire interface 420. In other embodiments, more or fewer fasteners 476 may be used. The aligned openings in the forward walls 220, 248 are positioned to correspond with one another when the modules are drawn into abutment by the winch 424, ensuring proper alignment for fastener installation.

The mechanical fastening system provides structural integrity to the assembled mobile structure 200 by creating secure mechanical coupling between the two separate modules at the interface 420. The fasteners 476 resist separation forces and maintain the structural connection between the first module 204 and the second module 232 during operation of the medical scanner 300. The distribution of multiple fasteners 476 across the interface 420 spreads the structural loads across the connection zone, preventing localized stress concentrations that could compromise the integrity of the assembled mobile structure 200.

The fastening system works in conjunction with the gasket 440 and the sealing plates to create a complete connection system at the interface 420. The fasteners 476 provide the mechanical strength to maintain the structural connection, while the gasket 440 provides environmental sealing, and the side sealing plate 464, top sealing plate 472, and corner sealing plate 468 provide exterior continuity and additional environmental protection. The combination of mechanical fastening and environmental sealing creates a robust connection system that maintains the structural and environmental integrity of the assembled mobile structure 200.

Referring to FIG. 8, a ground installation site is prepared for receiving the mobile structure 200. The ground surface 10 is formed by a poured slab that provides a stable foundation for the mobile structure 200. A bunker 14 is constructed on the ground surface 10 to accommodate the first module 204 during installation and operation. The bunker 14 is formed by concrete blocks 18 arranged to create sidewalls that define a recessed area within the installation site.

The concrete blocks 18 are stacked to form walls that surround at least three sides of the installation area where the first module 204 will be positioned. The bunker 14 has sidewalls having a thickness of at least two feet and a height of at least twelve feet. In some embodiments, the bunker sidewalls are formed 13 feet high and 2 feet thick surrounding substantially the entire first module 204. The substantial height and thickness of the walls formed by the concrete blocks 18 create a shielded space within which the first module 204 housing the medical scanner 300 can be positioned during operation.

The configuration of the bunker 14 provides radiation shielding around the first module 204 while maintaining accessibility for installation and maintenance operations. A gap exists between where the first module 204 will be positioned and the interior surfaces of the bunker 14, allowing a person to walk between the bunker sidewalls and the first module 204. The walkable gap provides access around the perimeter of the first module 204 for installation procedures and ongoing maintenance activities.

The ground surface 10 extends beyond the bunker 14, providing additional area for placement of the second module 232 during the assembly process. The poured slab forming the ground surface 10 accommodates the securing of both modules to the installation site.

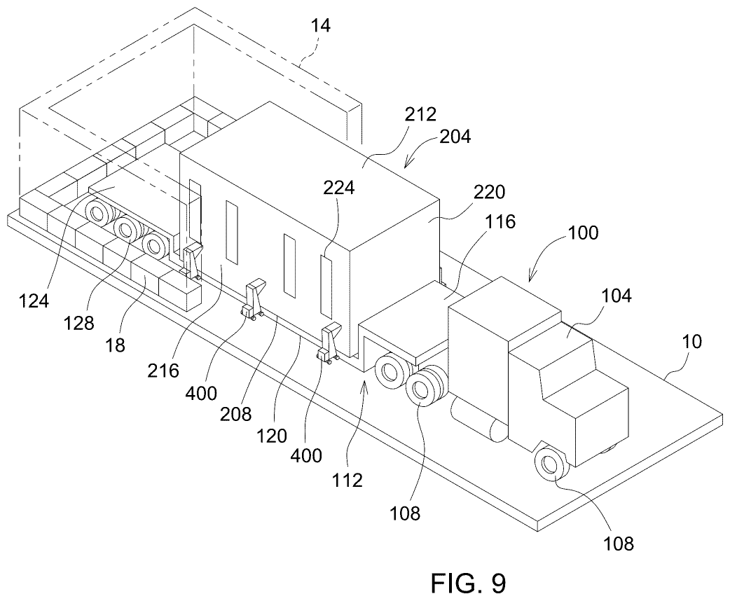

Referring to FIG. 9, a semi-trailer truck 100 carries the first module 204 to the installation site and backs into the bunker 14 for positioning and unloading operations. The semi-trailer truck 100 includes a tractor 104 with engine and cab that provides the motive power and operator control for the transport vehicle. The tractor 104 is supported by wheels 108 that enable highway transport and maneuvering at the installation site. The removable gooseneck trailer 112 connects to the tractor 104 through the gooseneck 116, which provides the articulating connection between the tractor 104 and the trailer assembly.

The removable gooseneck trailer 112 includes the main deck 120 that supports the first module 204 during transport and positioning operations. The rear deck 124 provides additional structural support for the trailer assembly and distributes the load of the first module 204 across the trailer structure. The removable gooseneck trailer 112 is supported by wheels 128 that bear the weight of the trailer and the first module 204 during transport and positioning operations.

The first module 204 is positioned on the main deck 120 of the removable gooseneck trailer 112, with the base 208 resting on the deck surface. The sidewalls 216 extend upward from the base 208 to define the exterior boundaries of the first module 204 and the transported module is sealed by a ceiling/roof 212 and a closed forward wall 220. In the illustrated embodiment, the first module includes one or more windows 224. The forward wall 220 faces toward the open area of the installation site where the second module 232 will be positioned during assembly operations.

Hydraulic jacking units 400 are positioned adjacent to the first module 204 to facilitate the removal of the first module 204 from the removable gooseneck trailer 112. The hydraulic jacking units 400 provide the lifting capability to raise the first module 204 vertically off the main deck 120 of the trailer, creating clearance between the base 208 of the first module 204 and the deck surface of the removable gooseneck trailer 112. The hydraulic jacking units 400 form part of a six-point portable lifting system that enables lifting and positioning of the first module 204 without requiring a crane. The six-point portable lifting system is carried on the same transport trailer that carries the first module 204, eliminating the need for separate lifting equipment to be transported to the installation site. The lifting system requires very little additional space beyond the footprint of the removable gooseneck trailer 112 and is simple to operate through hydraulic controls.

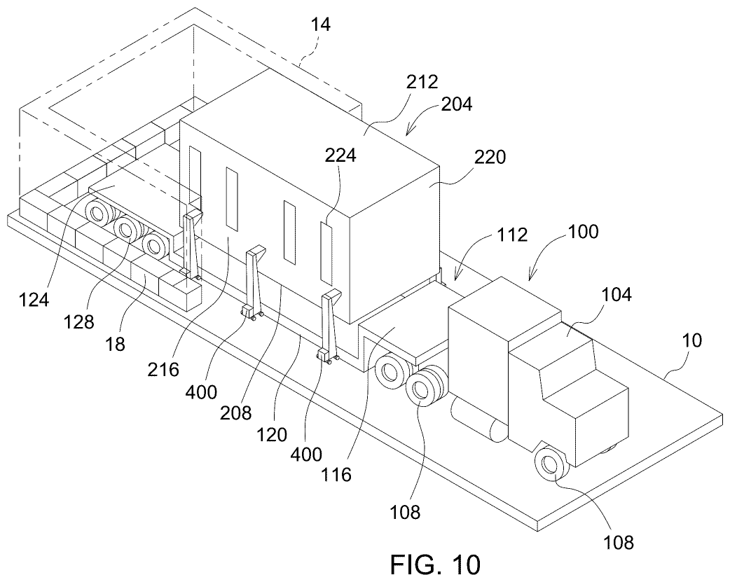

Referring to FIG. 10, the hydraulic jacking units 400 engage with the base 208 and or sidewalls 216 of the first module 204 and lift the first module 204 vertically upward from the main deck 120. The lifting operation raises the base 208 of the first module 204 above the main deck 120 and rear deck 124 of the removable gooseneck trailer 112, supporting the first module 204 at an elevated position above the trailer deck. The hydraulic jacking units 400 maintain the first module 204 in the elevated position while the semi-trailer truck 100 is driven away from the installation site.

Caster wheels 404 may be attached to the first module 204 via attachment points 408. The hydraulic jacking units 400 lower the first module 204 on the caster wheels 404 such that the first module 204 is rolled into its final position within the bunker 14. Once positioned within the bunker 14, the hydraulic jacking units 400 once again lift the first module 204 to allow for removal of the caster wheels 404 and attachment points 408. In some embodiments, these same attachment points 408 and caster wheels 404 will be reused with the second module 232.

The hydraulic jacking units 400 lower the first module 204 from the elevated position to its final operational position on the ground surface 10. The lowering operation positions the base 208 of the first module 204 in direct contact with the ground surface 10, with no intervening structure between the base 208 and the poured slab that forms the ground surface 10. The first module 204 rests directly on the ground surface 10, creating a stable foundation for the medical scanner 300 and associated equipment housed within the first module 204.

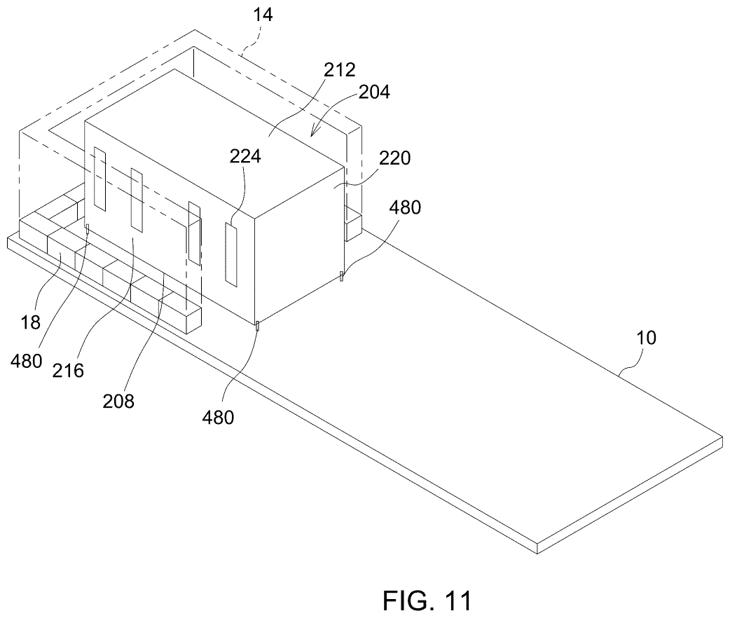

Referring to FIG. 11, the first module 204 is positioned within the bunker 14 in its final operational configuration. The bunker 14 extends upward from the ground surface 10 to a height that exceeds the height of the first module 204, providing radiation shielding around the perimeter of the first module 204. The concrete blocks 18 that form the bunker 14 create sidewalls that surround at least three sides of the first module 204, with the bunker sidewalls extending substantially above the roof 212 of the first module 204.

A gap exists between the sidewalls 216 of the first module 204 and the interior surfaces of the bunker 14, allowing access around the perimeter of the first module 204. The gap provides sufficient space for a person to walk between the bunker sidewalls and the exterior surfaces of the first module 204, enabling access for maintenance operations and inspection activities. The forward wall 220 of the first module 204 faces outward from the bunker 14. The forward wall 220 is a reusable panel that will be removed in a following step to provide the open end for connection to the second module 232 during assembly operations.

The first module 204 is secured to the ground surface 10 through a mounting system 480 that anchors the module to the poured slab. In some embodiments, the mounting system 480 includes a mounting anchor coupled to the first module 204 and a fastener extending through the mounting anchor to couple the first module 204 to the poured slab. The mounting anchor provides a connection interface between the structural framework of the first module 204 and the fastener that penetrates into the poured slab. The fastener creates a mechanical connection that prevents movement of the first module 204 relative to the ground surface 10 during operation of the medical scanner 300. By fastening the first module 204, the first module 204 is also prevented from movement upon use of the winch 424.

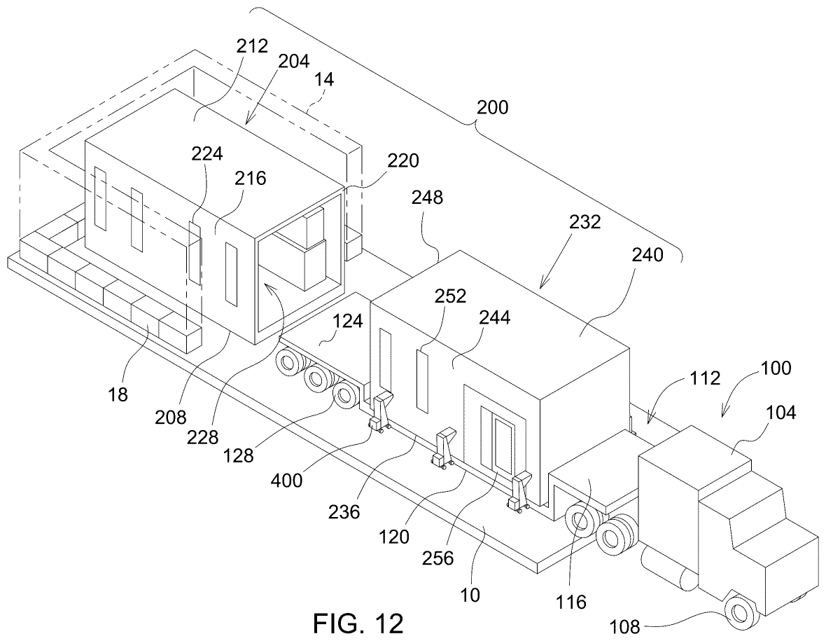

Referring to FIG. 12, the second module 232 is transported to the installation site on a semi-trailer truck 100 for positioning adjacent to the first module 204. The semi-trailer truck 100 and removable gooseneck trailer 112 are similar to the one used to transport the first module 204.

The second module 232 is enclosed by sidewalls 244 and a roof 240 and a door 256 is positioned within one of the sidewalls 244 to provide access to the interior spaces of the second module 232. The second module 232 is positioned on the main deck 120 of the removable gooseneck trailer 112 during the transport and positioning operations.

The semi-trailer truck 100 backs the removable gooseneck trailer 112 carrying the second module 232 until the second module 232 is positioned adjacent to the first module 204. The positioning operation places the open end of the second module 232 facing the open end of the first module 204, establishing the proper orientation for assembly operations. The second module 232 is initially positioned approximately 11 feet away from the first module 204 during this assembly step, with the spacing determined by the structural configuration of the removable gooseneck trailer 112 (i.e., the size of the rear deck 124) and the positioning requirements for subsequent assembly operations.

Reusable transport panels are removed from both the first module 204 and the second module 232 after the positioning operation. The reusable transport panels seal the open ends of each module during transport operations, providing environmental protection and structural closure for the interior spaces. The transport panels are configured for removal prior to assembly operations, exposing the open ends of both modules and providing access to the interface 420 where the modules will be joined together.

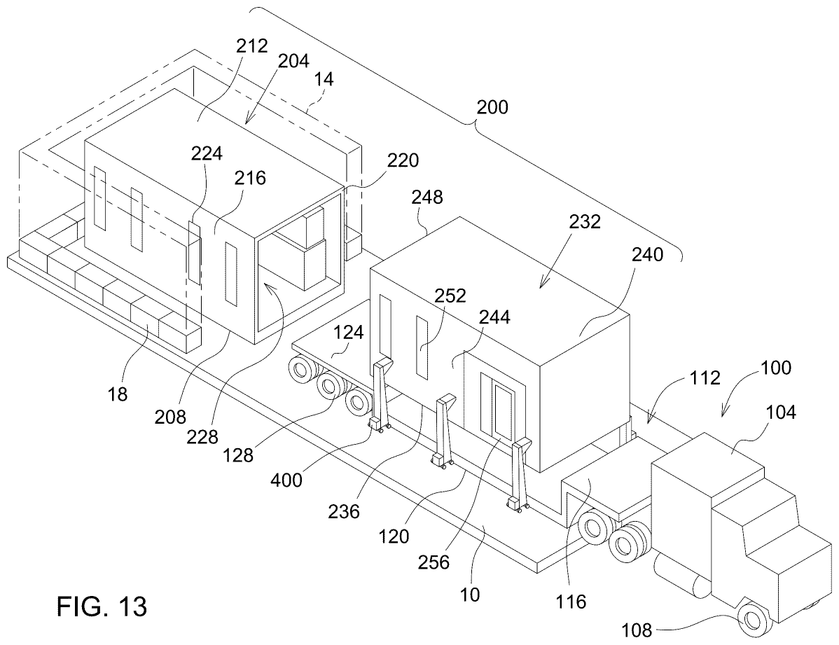

Referring to FIG. 13, the hydraulic jacking units 400 lift the second module 232 vertically off the removable gooseneck trailer 112, creating clearance between the second module 232 and the main deck 120. The lifting operation raises the second module 232 above the trailer deck, allowing the semi-trailer truck 100 to drive away from the installation site while the second module 232 remains suspended above the ground surface 10. The hydraulic jacking units 400 support the second module 232 at an elevated position above the ground surface 10, maintaining the second module 232 in position for subsequent assembly operations. The removal of the removable gooseneck trailer 112 leaves the second module 232 positioned above the concrete pad that forms the ground surface 10, with the second module 232 supported entirely by the hydraulic jacking units 400.

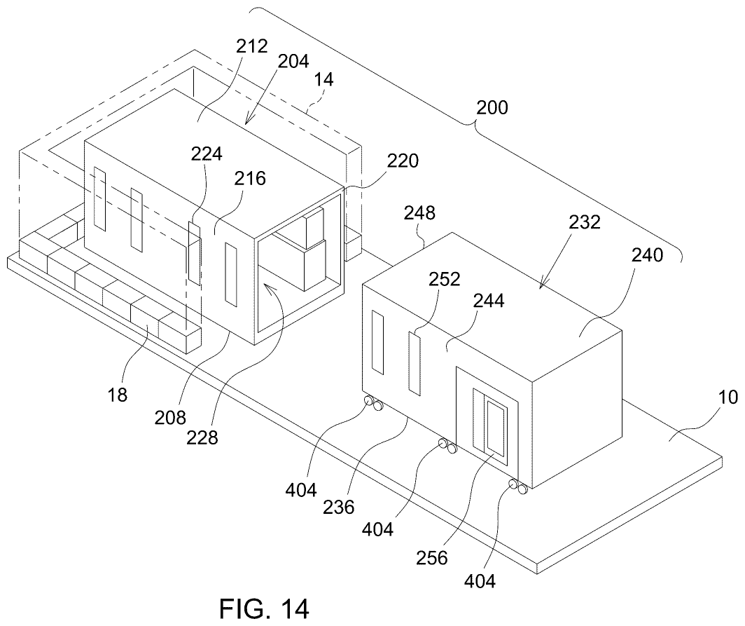

Referring to FIG. 14, caster wheels 404 are installed onto the second module 232 via the attachment points 408 to enable rolling movement of the second module 232 toward the first module 204. The caster wheels 404 couple to the attachment points 408 positioned along the perimeter of the second module 232, providing a temporary mobility system for the assembly process. The attachment points 408 accommodate the mounting of the caster wheels 404 through the rotational installation system described above, allowing the caster wheels 404 to be securely attached to the structural framework of the second module 232.

The caster wheels 404 support the second module 232 above the ground surface 10, enabling the second module 232 to be rolled along the concrete pad toward the first module 204. The rolling movement reduces the physical effort required to position the second module 232 and provides controlled movement during the assembly process. The second module 232 is moved on the caster wheels 404 until the second module 232 is positioned approximately six inches away from the first module 204. The positioning of the second module 232 at approximately six inches from the first module 204 establishes the proper spacing for the final assembly operation using the winch 424. The reduced gap between the modules minimizes the distance that the winch 424 must draw the second module 232 during the final positioning operation.

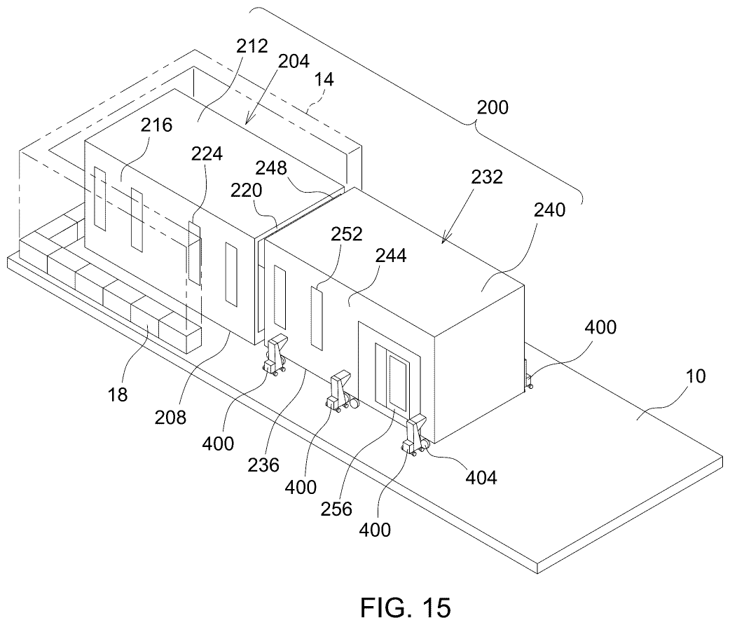

Referring to FIG. 15, the hydraulic jacking units 400 lift the second module 232 to enable removal of the caster wheels 404 from the attachment points 408. The lifting operation raises the second module 232 above the ground surface 10, creating clearance between the caster wheels 404 and the concrete pad. The elevated position of the second module 232 allows assembly personnel to access the attachment points 408 and remove the caster wheels 404 through the rotational removal process described above.

After removal of the caster wheels 404, the hydraulic jacking units 400 lower the second module 232 to position the base 236 directly on the ground surface 10. The lowering operation places the second module 232 in direct contact with the concrete pad, eliminating the gap that existed when the second module 232 was supported by the caster wheels 404. The second module 232 rests on the ground surface 10 at a distance of approximately six inches from the first module 204, establishing the final positioning for winch operation.

The winch 424 operates to draw the second module 232 toward the first module 204 through retraction of the cable 432 connected to the attachment point 408 in the second module 232. The winch operation pulls the second module 232 across the remaining six-inch gap between the modules, drawing the second module 232 into direct abutment with the first module 204 at the interface 420. The guide plates 436 direct the sidewalls 244 of the second module 232 into proper alignment with the sidewalls 216 of the first module 204 during the drawing operation.

The winch operation continues until the forward wall 248 of the second module 232 contacts the forward wall 220 of the first module 204, creating direct abutment between the two modules. The chain binder 444 may be utilized if necessary to provide additional alignment transverse to the drawing direction of the winch 424. The gasket 440 positioned at the interface 420 compresses between the mating surfaces of the modules, forming an environmental seal around the perimeter of the connection zone. The completed winch operation positions the first module 204 and the second module 232 in direct contact, forming an enclosed space defined by the combined interior volumes of both modules.

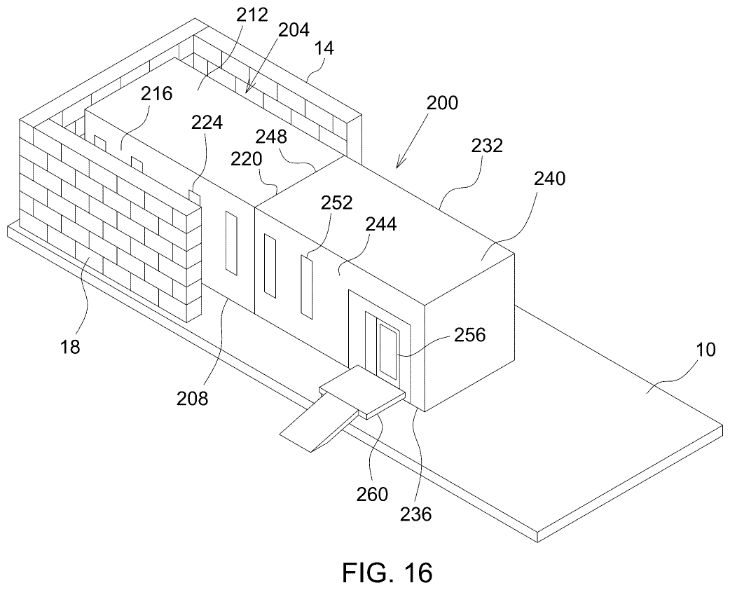

Referring to FIG. 16, the mobile structure 200 is shown in its assembled configuration positioned on the ground surface 10. The first module 204 and the second module 232 are joined together to form a complete facility, with the sealing plates installed to provide structural continuity and environmental protection at the interface 420. The side sealing plate 464, top sealing plate 472, and corner sealing plate 468 create a comprehensive sealing system around the perimeter of the interface 420, bridging the connection between the modules and forming a unified exterior envelope for the assembled mobile structure 200.

The second module 232 is secured to the ground surface 10 through a mounting system similar to that used for the first module 204. A mounting anchor is coupled to the second module 232 and a fastener extends through the mounting anchor to couple the second module 232 to the poured slab that forms the ground surface 10. The mounting anchor provides a connection interface between the structural framework of the second module 232 and the fastener that penetrates into the concrete pad. The fastener creates a mechanical connection that prevents movement of the second module 232 relative to the ground surface 10 during operation of the medical scanner 300 and other office equipment and medical device control devices 304.

A ramp 260 is added to the exterior of the mobile structure 200 to provide access to the door 256 positioned within the sidewalls of the second module 232. The ramp 260 extends from the ground surface 10 to the door 256, creating an inclined pathway that accommodates wheelchair access and stretcher movement into the mobile structure 200. The direct positioning of the mobile structure 200 on the ground surface 10 minimizes the height differential between the ground surface 10 and the door 256, requiring only a minimal step up from the ground surface 10 to access the interior spaces of the mobile structure 200. The ramp 260 height is minimized to account only for the thickness of the base 236 of the second module 232, providing easy access for elderly patients, patients with disabilities, and patients transported in wheelchairs or on stretchers.

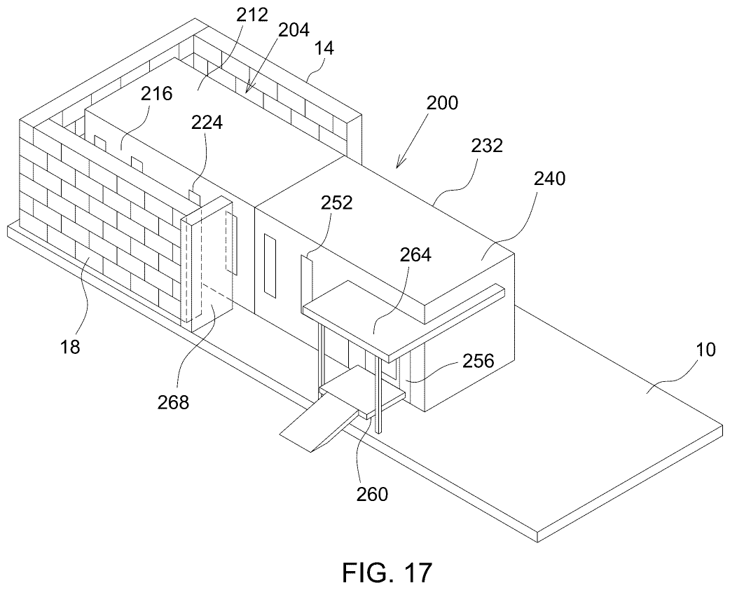

Referring to FIG. 17, the mobile structure 200 is shown in the final assembly configuration with additional exterior components installed to complete the installation. An awning 264 is positioned above the ramp 260 to provide weather protection for patients and personnel accessing the door 256 of the second module 232. The awning 264 extends outward from the mobile structure 200 over the ramp 260, creating a sheltered area. Sheathing 268 is installed around the perimeter of the bunker 14 to prevent unauthorized access into the space between the first module 204 and the concrete blocks 18 that form the bunker sidewalls, while maintaining the accessibility for authorized maintenance and inspection activities. The final assembly configuration demonstrates the complete mobile structure 200 positioned directly on the ground surface 10 with minimal elevation above the concrete pad. The ramp 260 provides accessible entry to the door 256 with a minimal incline, accommodating wheelchair users and stretcher transport with reduced physical barriers.

A number of implementations have been described. Nevertheless, it will be understood that various modifications may be made without departing from the spirit and scope of the disclosure. Accordingly, other implementations are within the scope of the following claims.

1. A method of assembling a mobile structure having a medical scanning device, the method comprising:
locating a first portion of the mobile structure on a ground surface, the first portion of the mobile structure having the medical scanning device mounted therein;
securing the first portion to the ground surface;
positioning a second portion of the mobile structure relative to the first portion such that an open end of the first portion is positioned adjacent to an open end of the second portion;
operating a winch to draw the second portion into abutment with the first portion such that the first portion and the second portion form an enclosed space defined by the first portion and the second portion; and
securing the second portion to the ground surface.

2. The method of claim 1, wherein the ground surface is a poured slab, and wherein securing the first portion to the ground surface includes providing a mounting system having a mounting anchor and a fastener to couple the first portion to the poured slab.

3. The method of claim 2, wherein securing the second portion to the ground surface includes providing a fastener through a mounting anchor coupled to the second portion and into the poured slab.

4. The method of claim 1, wherein the mobile structure includes a floor surface upon which a user is able to walk within the enclosed space, wherein the winch is located below the floor surface.

5. The method of claim 4, wherein the winch is coupled to one of the first portion or the second portion and an attachment point is coupled to the other of the first portion or the second portion, wherein operating the winch includes coupling a cable of the winch to the attachment point.

6. The method of claim 5, wherein the attachment point is located below the floor surface.

7. The method of claim 1, further comprising positioning a first guide plate on a first wall of the first portion and a second guide plate on a second wall of the first portion, wherein the first and second guide plates guide a first wall and a second wall of the second portion towards the first wall and the second wall of the first portion during operation of the winch.

8. The method of claim 7, wherein the first guide plate extends outward from an end of the first wall of the first portion at an angle of 15-45 degrees and a distance of 4-12 inches.

9. The method of claim 1, further comprising aligning the first portion and the second portion with a chain binder.

10. The method of claim 1, further comprising coupling casters to the second portion and moving the second portion towards the first portion on the casters prior to operating the winch.

11. The method of claim 10, further comprising removing the casters prior to operating the winch.

12. The method of claim 1, further comprising removing the first portion from a wheeled trailer prior to locating the first portion of the mobile structure on the ground surface and removing the second portion from a wheeled trailer prior to positioning the second portion of the mobile structure relative to the first portion.

13. The method of claim 1, wherein locating the first portion of the mobile structure on the ground surface includes locating the first portion within a bunker having sidewalls that surround at least three sides of the first portion.

14. The method of claim 13, wherein the bunker has sidewalls having a thickness of at least two feet and a height of at least twelve feet.

15. The method of claim 1, wherein locating the first portion of the mobile structure on the ground surface includes locating the first portion directly on the ground surface with no intervening structure.

16. A mobile structure having a medical scanning device, the mobile structure comprising:
a first portion having a floor surface and a plurality of walls, the medical scanning device mounted to the floor surface of the first portion, the first portion configured to be transported on a trailer;
a second portion having a floor surface and a plurality of walls, the second portion formed separate from the first portion and configured to be transported on a trailer separate from the first portion; and
a winch located below the floor surface of the first portion, the winch having a cable configured to couple to an attachment point of the second portion, wherein the winch is configured to draw the second portion into abutment with the first portion.

17. The mobile structure of claim 16, further comprising a first mounting anchor coupled to the first portion to couple the first portion to a poured slab, and a second mounting anchor coupled to the second portion to couple the second portion to the poured slab.

18. The mobile structure of claim 16, further comprising a first guide plate coupled to a first wall of the plurality of walls of the first portion and a second guide plate coupled to a second wall of the plurality of walls of the first portion, wherein the first guide plate and the second guide plate are configured to align the plurality of walls of the first portion with the plurality of walls of the second portion.

19. The mobile structure of claim 16, further comprising caster wheels coupled to the second portion such that the second portion is movable relative to the first portion.

20. A mobile structure having a medical scanning device, the mobile structure comprising:
a first portion and a second portion collectively forming an enclosed space configured to support the medical scanning device, a patient, and an operator of the medical scanning device.
